(19) United States
(12) Patent Application Publication
(10) Pub. No.: US 2021/0102026 A1
(43) Pub. Date: Apr. 8, 2021

(54) N,N'-DIALKYL METHYLCYCLOHEXANEDIAMINE AS REACTIVE DILUENT WITHIN EPOXY RESIN SYSTEMS

(71) Applicant: BASF SE, Ludwigshafen am Rhein (DE)

(72) Inventors: Matthaeus KOPCZYNSKI, Ludwigshafen (DE); Alexander PANCHENKO, Ludwigshafen (DE); Irene GORMAN, Ludwigshafen (DE); Michael HENNINGSEN, Ludwigshafen (DE)

(73) Assignee: BASF SE, Ludwigshafen am Rhein (DE)

(21) Appl. No.: 16/627,505

(22) PCT Filed: Jul. 13, 2018
(86) PCT No.: PCT/EP2018/069060

§ 371 Date: Dec. 30, 2019

(30) Foreign Application Priority Data
Jul. 25, 2017 (EP) 17183013.6

(51) Int. Cl. C08G 59/50; C08G 59/56; C07C 211/36

(52) U.S. Cl. CPC C08G 59/5026; C07C 2601/14; C07C 211/36; C08G 59/56

(57) ABSTRACT

Secondary diamine N,N′-dialkyl methylcyclohexanediamine acts as a reactive diluent for curable epoxy resin compositions. The addition of this compounds significantly reduces the initial viscosity of the epoxy resin composition while the resulting cured epoxy resin exhibits comparable favorable mechanical, chemical resistance and thermal properties such as low water uptake and high glass transition temperatures. Such compositions are particular suitable for manufacturing of composites with high mechanical and heat resistance properties by the means of resin transfer molding (RTM), vacuum aided resin transfer molding (VARTM) or infusion technology.

The subject matter of the present invention relates to a hardener component for curing of epoxy resins, comprising at least one curing agent and N,N′-dialkyl methylcyclohexanediamine as a reactive diluent. Accordingly the subject matter of the present invention also relates to an epoxy resin composition comprising (i) a resin component comprising at least one epoxy resin and (ii) a hardener component comprising N,N′-dialkyl methylcyclohexanediamine and at least one curing agent. The invention also relates to a process for producing said epoxy resin composition, to the use of the epoxy resin composition of the invention for producing cured epoxy resin, and to a cured epoxy resin made of the epoxy resin composition of the invention. Finally, the invention also relates to the use of N,N′-dialkyl methylcyclohexanediamine as a reactive diluent with epoxy resin compositions.

Epoxy resins are generally known and due to their toughness, flexibility, adhesion, and chemicals resistance, are used widely in coating, adhesives, molding and lamination materials, as well as for the manufacture of fiber-reinforced composite materials.

Recently, hardened epoxy resins reinforced with carbon or glass fiber have become particularly important to produce rotor blades in wind turbine construction. Due to the large size of these parts, problem free impregnation of the reinforcing fibers is of utmost importance. For epoxy systems, this means a long open time (pot life) where viscosity remains low and gelation has not occurred. When epoxy systems are too reactive, the viscosity can reach a state where injection is no longer possible and injection must stop, even though the mold is not completely filled.

Starting from epoxy compounds having at least two epoxy groups it is possible by way of example to use an amino compound having two amino groups for curing via polyaddition reaction (chain extension). High-reactivity amino compounds are generally added only briefly prior to the desired curing. The systems are therefore what are known as 2-component (2C) systems. In principle, amine curing agents are classified in accordance with their chemical structure into aliphatic, cycloaliphatic, or aromatic types.

Viscosity of an epoxy system can be reduced by a diluent. Diluents are typically employed to reduce viscosity, but may also be selected to extend pot life. The diluents can be either un-reactive or reactive. Typically, un-reactive diluents include benzyl alcohols, glycols, and alkyl phenols. However, these un-reactive diluents are not incorporated into the crosslinked network, and formulations employing this class of diluents typically suffer from significant losses in mechanical and thermal properties, as well as high VOC emissions. Alternatively, reactive diluents can be employed. Epoxy-type reactive diluents will contain one or more epoxy moieties, and classic examples include, monoglycidyl ether of C12-C14 alcohol and diglycidyl ether of 1,4-butanediol, with the mono-functional species being more effective diluents. However, the use of both mono and di-functional reactive diluents usually result in a decrease in the mechanical, thermal, and chemical resistance properties, with these effects being more pronounced for mono-functional species. Furthermore, systems incorporating high levels of butanediol based diluents suffer from increased water uptake. Lastly, mono- and di-epoxide diluents tend to be more powerful skin-sensitizing agents, as compared to the standard bisphenol-A based epoxy resin.

As described by Henry Lee and Kris Neville in Handbook of Epoxy Resins, epoxy functional reactive diluents are synthesized through the reaction of aliphatic alcohol and epichlorohydrin to from a chlorine intermediate, which is then de-halogenated to generate oxirane rings. However, the aliphatic hydroxyl group found on the chlorine intermediated of aliphatic alcohols reacts at a similar rate to the aliphatic alcohol on the polyol starting material. This similarity in reactivity results in a relatively high amount of both hydrolysable and organic chlorine, caused by side reactions, in the final product. Therefore, epoxy functional reactive diluents suffer from excess chlorine content, which can be detrimental to electrical properties, color, and reactivity.

U.S. Pat. Nos. 5,426,157 and 5,739,209 describe epoxy resin composition where secondary amines are incorporated into the hardener mixture to impart improved flexibility and resistances. U.S. Pat. No. 6,642,344 describes cycloaliphatic secondary diamines based on cyclohexane, again with the goal of imparting flexibility, for use with an epoxy curing agent or with an epoxy curing agent mixture. This patent refers to the ability of these secondary amines to increase the curing time, however, there is no mention as to a viscosity diluting effect. US 20120226017 A describes the preparation of a stereoisomer mixture of methylcyclohexane diamines by hydrogenation of 2,4- and 2,6-toluenediamine and its use as curing agent for epoxy resins. CN 106083607 A describes the preparation of N,N′-dialkyl methylcyclohexanediamines and its use for slow resin curing. CN 103524717 A describes the modification of methylcyclohexanediamines with acrylonitrile and the use of the modified compound as slow curing agent. US 20150344406 A describes the use of a secondary amine, 1,3-bis(2-ethylhexylaminomethyl)-benzene, as a reactive diluent in low-emission coatings, coverings, and paints application. However, this secondary amine allows for only moderate reduction of the initial viscosity of epoxy systems.

An object underlying the invention can therefore be considered to be the provision of reactive diluents for curable compositions, which are essentially free of organic chlorine and allow for low initial viscosity, and which exhibit after curing comparable favorable mechanical, chemical resistance and thermal properties such as low water uptake and high glass transition temperatures. Preferably, such a curable composition features comparatively long available operating time with comparatively low viscosity, even when exposed to higher curing temperatures. Such compositions are particular suitable for manufacturing of composites with high mechanical and heat resistance properties by the means of resin transfer molding (RTM), vacuum aided resin transfer molding (VARTM) or infusion technology. It is therefore the object of the present invention to provide a reactive diluent which allows for viscosity dilution and reactivity dilution without the drawbacks of classic aliphatic epoxy reactive diluents or un-reactive diluents.

The inventors found that N,N′-dialkyl methylcyclohexanediamine acts as a favorable reactive diluent when added to the hardener component of an epoxy resin composition. It can effectively dilute epoxy resin compositions in a similar way to standard epoxy-based reactive diluents, without detrimental effects on water uptake, mechanical and thermal properties or chlorine content. For the purposes of the invention, reactive diluents are compounds which reduce the initial viscosity of the curable composition and which, during the course of the curing of the curable composition, enter into chemical bonding with the network as it forms from the curable composition.

Accordingly, the present invention provides a composition (“hardener component”) for curing epoxy resins comprising N,N′-dialkyl methylcyclohexanediamine and at least one curing agent, wherein the at least one curing agents are amino hardeners having at least one primary aliphatic amine group and an NH-functionality of at least 3.

Also, the present invention provides a composition (“epoxy resin composition”) comprising the hardener component of the invention and a resin component, which comprises at least one epoxy resin.

Epoxy resins according to this invention usually have from 2 to 10, preferably from 2 to 6, very particularly preferably from 2 to 4, and in particular 2, epoxy groups. The epoxy groups are in particular the glycidyl ether groups that are produced in the reaction of alcohol groups with epichlorohydrin. The epoxy resins can be low-molecular-weight compounds which generally have an average molar mass (Mn) smaller than 1000 g/mol or relatively high-molecular-weight compounds (polymers). These polymeric epoxy resins preferably have a degree of oligomerization of from 2 to 25, particularly preferably from 2 to 10, units. They can be aliphatic or cycloaliphatic compounds, or compounds having aromatic groups. In particular, the epoxy resins are compounds having two aromatic or aliphatic 6-membered rings, or oligomers thereof. Epoxy resins important in industry are obtainable via reaction of epichlorohydrin with compounds which have at least two reactive hydrogen atoms, in particular with polyols. Particularly important epoxy resins are those obtainable via reaction of epichlorohydrin with compounds comprising at least two, preferably two, hydroxy groups and comprising two aromatic or aliphatic 6-membered rings. Compounds of this type that may in particular be mentioned are bisphenol A and bisphenol F, and also hydrogenated bisphenol A and bisphenol F—the corresponding epoxy resins being the diglycidyl ethers of bisphenol A or bisphenol F, or of hydrogenated bisphenol A or bisphenol F. Bisphenol A diglycidyl ether (DGEBA) is usually used as epoxy resin according to this invention. Other suitable epoxy resins according to this invention are tetraglycidylmethylenedianiline (TGMDA) and triglycidylaminophenol, and mixtures thereof. It is also possible to use reaction products of epichlorohydrin with other phenols, e.g. with cresols or with phenol-aldehyde adducts, for example with phenol-formaldehyde resins, in particular with novolaks. Other suitable epoxy resins are those which do not derive from epichlorohydrin. It is possible to use, for example, epoxy resins which comprise epoxy groups via reaction with glycidyl (meth)acrylate. It is preferable in the invention to use epoxy resins or mixtures thereof which are liquid at room temperature (25° C.). The epoxy equivalent weight (EEW) gives the average mass of the epoxy resin in g per mole of epoxy group.

It is preferable that the curable composition of the invention is composed of at least 30%, preferably at least 50% by weight of epoxy resin.

In a particular embodiment of the invention, the epoxy resin component comprises—in addition to the at least on epoxy resin—one or more reactive diluents which have functional groups, which are able to react with the hydroxyl groups of the resin and/or with the functional groups of the curing agent, with formation of covalent bonds. Such reactive diluents are for example selected from the group consisting of ethylene carbonate, vinylene carbonate, propylene carbonate, 1,4-butanediol bisglycidyl ether, 1,6-hexanediol bisglycidyl ether (HDBE), glycidyl neodecanoate, glycidyl versatate, 2-ethylhexyl glycidyl ether, neopentyl glycol diglycidyl ether, p-tert-butyl glycidic ether, butyl glycidic ether, C8-C10-alkyl glycidyl ether, C12-C14-alkyl glycidyl ether, nonylphenyl glycidic ether, p-tert-butyl phenyl glycidic ether, phenyl glycidic ether, o-cresyl glycidic ether, polyoxypropylene glycol diglycidic ether, trimethylolpropane triglycidic ether (TMP), glycerol triglycidic ether, triglycidyl-para-aminophenol (TGPAP), divinylbenzyl dioxide and dicyclopentadiene diepoxide. They may make up a proportion of up to 30% by weight, particularly up to 25% by weight, in particular from 1 to 20% by weight, based on the total amount of epoxy resin of the epoxy resin composition. In a particular embodiment of the invention, such reactive diluents make up a portion of less than 5% by weight, preferably less than 2% by weight based on the total amount of epoxy resin of the epoxy resin composition. In a further particular embodiment of the invention, the epoxy resin composition is in essence free from such reactive diluents, preferably, the epoxy resin composition is free from such reactive diluents.

The expression “in essence free” means for the purposes of this invention a proportion 1% by weight, preferably 0.1% by weight, particularly preferably “below the detection threshold”, based on the corresponding entire composition.

It is preferable that the N,N′-dialkyl methylcyclohexanediamine is N,N′-dialkyl 4-methylcyclohexane-1,3-diamine of the general formula I

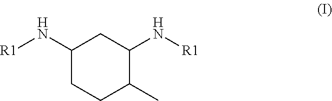

or N,N′-dialkyl 2-methylcyclohexane-1,3-diamine of formula II

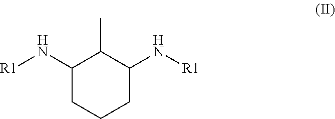

or a mixture thereof, where each

R1 is independently of each other and is an alkyl group having from 1 to 4 carbon atoms, particularly 3 to 4 carbon atoms. With very particular preference more particularly, the radical selected for R1 is an aliphatic hydrocarbon radical selected from the group consisting of methyl, ethyl, n-propyl, isopropyl, n-butyl, iso-butyl, and sec-butyl, more preferably from the group consisting of isopropyl and sec-butyl.

In a preferred embodiment of the invention, N,N′-dialkyl methylcyclohexanediamine is N,N′-diisopropyl 4-methylcyclohexane-1,3-diamine, N,N′-diisopropyl 2-methylcyclohexane-1,3-diamine, or a mixture thereof. Preferably N,N′-dialkyl methylcyclohexanediamine is a mixture of N,N′-diisopropyl 4-methylcyclohexane-1,3-diamine and N,N′-diisopropyl 2-methylcyclohexane-1,3-diamine. In a preferred mixture of N,N′-diisopropyl 4-methylcyclohexane-1,3-diamine and N,N′-diisopropyl 2-methylcyclohexane-1,3-diamine, the proportion of N,N′-diisopropyl 4-methylcyclohexane-1,3-diamine is in the range of 60 to 95% by weight, preferably in the range of 70 to 90% by weight, particularly in the range of 75 to 85% by weight, and correspondingly the proportion of N,N′-diisopropyl 2-methylcyclohexane-1,3-diamine is in the range of 5 to 40% by weight, preferably in the range of 10 to 30% by weight, particularly in the range of 15 to 25% by weight.

By way of example, N,N′-dialkyl methylcyclohexanediamine can be produced from the corresponding amino arenes (diamino toluenes) pursuant to Oh et al. (Catalysis Comm. (43 (2014), 79-83) or from the corresponding primary amine (methylcyclohexanediamine) by converting the primary amine groups with a ketone or aldehyde in the presence of hydrogen. As described in WO2011/033104, 4-methylcyclohexane-1,3-diamine and 2-methylcyclohexane-1,3-diamine encompass several stereoisomers. Accordingly, also the corresponding secondary amines of the present invention, N,N′-dialkyl methylcyclohexanediamine, and in particular N,N′-dialkyl 4-methylcyclohexane-1,3-diamine and N,N′-dialkyl 2-methylcyclohexane-1,3-diamine, can be particular stereoisomers or mixtures thereof. In a particular embodiment of the invention, the N,N′-dialkyl methylcyclohexanediamine is produced from one of the mixtures of stereoisomers of 4-methylcyclohexane-1,3-diamine and 2-methylcyclohexane-1,3-diamine described in WO2011/033104.

For the purposes of the invention, alkyl groups have from 1 to 20 carbon atoms. They can be linear, branched, or cyclic. They can be saturated or (poly)unsaturated. They are preferably saturated. They have no substituents having heteroatoms. Heteroatoms are all atoms other than C and H atoms.

For epoxy resin compositions or hardener components of the invention, the N,N′-dialkyl methylcyclohexanediamine of the invention preferably makes up a proportion of up to 70% by weight, particularly preferably up to 60% by weight, in particular from 15 to 60% by weight, based on the total amount of N,N′-dialkyl methylcyclohexanediamine(s) and curing agent(s) of the hardener component.

For epoxy resin compositions of the invention, the N,N′-dialkyl methylcyclohexanediamine of the invention preferably makes up a proportion of up to 40% by weight, particularly preferably from 1 to 30% by weight, in particular from 3 to 25% by weight, based on the total amount of epoxy resin.

The Curing agents of the invention (amino hardeners) have the ability to crosslink epoxy resins, preferably bisphenol A diglycidyl ether (DGEBA), but which do not essentially react with the secondary amino groups of the N,N′-dialkyl methylcyclohexanediamine. Curing agents do not essentially react with the secondary amino groups of the N,N′-dialkyl methylcyclohexanediamine if <10%, preferably <5%, particularly <1% particularly preferably none of these secondary amino groups are converted within 24 h at room temperature (25° C.), preferably at 40° C., in particular at 60° C. Accordingly, curing agents can convert epoxy resins, e.g. bisphenol A diglycidyl ether (DGEBA), into three-dimensionally crosslinked thermoset materials (cured epoxy resins).

The at least one curing agent of the hardener component of the invention are amino hardeners having at least one primary aliphatic amine group and an NH-functionality of at least 3 (for example, at least one primary and one secondary amino group), more particularly those having two primary aliphatic amino groups (NH-functionality of 4).

The NH-functionality of an amino compound here corresponds to its number of NH bonds. A primary amino group therefore has a NH-functionality of 2, while a secondary amino group has an NH-functionality of 1. The linking of the amino groups of the amino hardener with the epoxide groups of the epoxy resin produces polymers from the amino hardener and the epoxy resin, the epoxide groups being reacted to form free OH groups. For the purposes of the invention, primary aliphatic amine groups are primary amine groups which are bound to a carbon atom which is not part of an aromatic or tautomeric system. Accordingly, dicyandiamide—by the way of an example—is no amino hardener in the meaning of this invention.

In a particular embodiment of the invention, the hardener component or the epoxy resin composition contains more than 95% by weight, preferably more than 98% by weight of amino hardener as curing agent based on the total of all employed curing agents. In a further particular embodiment of the invention, the epoxy resin composition is in essence free from curing agents other than amino hardeners, preferably, the epoxy resin composition is free from curing agents other than amino hardeners.

Further preferred curing agents for the purpose of the invention are amino hardeners selected from the group consisting of 2,2-dimethyl-1,3-propanediamine, 1,3-pentanediamine (DAMP), 1,5-pentanediamine, 1,5-diamino-2-methylpentane (MPMD), 1,6-hexanediamine, 1,7-heptanediamine, 1,8-octanediamine, 1,9-nonanediamine, 1,10-decanediamine, 1,11-undecanediamine, 1,12-dodecanediamine, 2,5-dimethyl-1,6-hexanediamine, 2,2,4- and 2,4,4-trimethylhexamethylenediamine (TMD), dimethyl dicykan (DMDC), isophoronediamine (IPDA), diethylenetriamine (DETA), triethylenetetramine (TETA), aminoethylpiperazine (AEP), meta-xylylene diamine (MXDA), styrene modified MXDA (Gaskamine 240), 1,3-bis(aminomethyl cyclohexane) (1,3-BAC), bis(p-aminocyclohexyl)methane (PACM), methylenedianiline (e.g. 4,4′-methylenedianiline), polyetheramines (e.g. polyetheramine D230, poly(glycol amine)), diaminodiphenylmethane (DDM), diaminodiphenylsulfone (DDS), 2,4-toluenediamine, 2,6-toluenediamine, methylcyclohexane-1,3-diamine (MCDA) (e.g. 4-methylcyclohexane-1,3-diamine, 2-methylcyclohexane-1,3-diamine, or mixtures thereof), diethyltoluenediamine (DETDA) (e.g. 2,4-diamino-3,5-diethyltoluene or 2,6-diamino-3,5-diethyltoluene, 1,2-diaminobenzene), 1,3-diaminobenzene, 1,4-diaminobenzene, diaminocyclohexane (e.g. 1,2-diaminocyclohexane (DACH)), 1,8-menthanediamine, diaminodiphenyl oxide, 3,3′,5,5′-tetramethyl-4,4′-diaminobiphenyl and 3,3′-dimethyl-4,4′-diaminodiphenyl, and also mixtures thereof.

Further preferred curing agents for the purpose of the invention are adducts formed from polyamines and epoxy resins or epichlorohydrin and having at least one primary aliphatic amine group and an NH-functionality of at least 3 (for example, at least one primary and one secondary amino group), more particularly those having two primary aliphatic amino groups (NH-functionality of 4).

For epoxy resin compositions of the invention, which comprise only amino hardeners as curing agent, it is preferred that the epoxy compounds of the resin component (epoxy resin(s) and, if any, epoxy based reactive diluent(s)) and the amino compounds of the hardener component (amino hardener(s) and N,N′-dialkyl methylcyclohexanediamine(s)) are preferably used in an approximately stoichiometric ratio in terms of the epoxide groups and NH-functionalities. Particularly suitable ratios of epoxide groups to NH-functionality are 1:0.8 to 0.8:1.

The hardener component or epoxy resin composition of the invention may also comprise an accelerator for the curing. Suitable curing accelerators are, for example, tertiary amines, imidazoles, imidazolines, guanidines, urea compounds, and ketimines. Suitable tertiary amines are, for example, N,N-dimethylbenzylamine, 2,4,6-tris(dimethylaminomethyl)phenol (DMP 30), 1,4-diazabicyclo[2.2.2]octane (DABCO), 1,8-diazabicyclo[5.4.0]undec-7-ene (DBU), S-triazine (Lupragen N 600), bis(2-dimethylaminoethyl) ether (Lupragen N 206), pentamethyldiethylenetriamine (Lupragen N 301), trimethylaminoethylethanolamine (Lupragen N 400), tetramethyl-1,6-hexanediamine (Lupragen N 500), aminoethylmorpholine, aminopropylmorpholine, aminoethylethyleneurea or N-alkyl-substituted piperidine derivatives. Suitable imidazoles are imidazole itself and its derivatives such as, for example, 1-methylimidazole, 2-methylimidazole, Nbutylimidazole, benzimidazole, N-C1-12-alkylimidazoles, N-arylimidazoles, 2,4-ethylmethylimidazole, 2-phenylimidazole, 1-cyanoethylimidazole or N-aminopropylimidazole. Suitable imidazolines are imidazoline itself and its derivatives such as, for example, 2-phenylimidazoline. Suitable guanidines are guanidine itself or its derivatives such as, for example, methylguanidine, dimethylguanidine, trimethylguanidine, tetramethylguanidine (TMG), methyl isobiguanide, dimethyl isobiguanide, tetramethyl isobiguanide, hexamethyl isobiguanide, heptamethyl isobiguanide or dicyandiamine (DICY). Suitable urea compounds are urea itself and its derivatives such as, for example, 3-(4-chlorophenyl)-1,1-dimethylurea (monuron), 3-phenyl-1,1-dimethylurea (fenuron), 3-(3,4-dichlorophenyl)-1,1-dimethylurea (diuron), 3-(3-chloro-4-methylphenyl)-1,1-dimethylurea (chlorotoluron), and tolyl-2,4-bis-N,N-dimethylcarbamide (Amicure UR2T). Suitable ketimines are, for example, Epi-Kure 3502 (a reaction product from ethylenediamine with methyl isobutyl ketone). Some of these accelerators also belong to the group of curing agents according to the invention since they act as curing agent and as accelerator.

The epoxy resin compositions of the invention can also comprise additives, for example inert diluents, reinforcing fibers (in particular glass fibers or carbon fibers), pigments, dyes, fillers, release agents, tougheners, flow agents, antifoams, flame-retardant agents, or thickeners. It is usual to add a functional amount of these additives, an example being a pigment in an amount which leads to the desired color of the epoxy resin compositions. The epoxy resin compositions of the invention usually comprise from 0 to 50% by weight, preferably from 0 to 20% by weight, for example from 2 to 20% by weight, of the entirety of all of the additives, based on the entire curable composition. For the purposes of this invention, the term additives means any of the additions to the curable composition which are neither epoxy compound nor reactive diluent nor curing agent.

The invention further provides a process for the production of cured epoxy resins made of the epoxy resin composition of the invention. In this process, the epoxy resin composition of the invention is provided and then cured. To this end, the components (resin component and hardener component and optionally other components, for example additives) are brought into contact with one another and mixed, and then cured at a temperature that, in terms of the application, is practicable. The suitable curing temperature depends on the employed curing agent. The curing process can take place at atmospheric pressure and at temperatures below 250° C., in particular at temperatures below 210° C., preferably at temperatures below 185° C., in particular in the temperature range from 0 to 210° C., very particularly preferably in the temperature range from 10 to 185° C.

The hardener component of the invention which comprises N,N′-dialkyl methylcyclohexanediamine and at least one amino hardener as curing agent, can be used—in addition to the curing of epoxy resins—also for the curing of polyisocyanates and derivatives thereof, such as toluene diisocyanate (TDI), methylene diphenyl diisocyante (MDI), polymeric methylene diphenyl diisocyante (PMDI), isophorone diisocyante (IPDI), hexamethylene diisocyante (HDI), dicyclohexylmethane diisocyante (H12MD1), as well as isocyanurates thereof, allophanates thereof, biurets thereof, and prepolymers thereof with aminic or alcoholic compounds, especially with polyetheramines (D230, D400, T403, D2000, T5000). Such curing of polyisocyanates and derivatives thereof can be carried out for example at a temperature in a range from −40° C. to 100° C. Preferably no additional catalysts are employed. Such polyisocyante based curing systems can be used for applications such as casting, spraying and molding.

The invention particularly provides a process for the production of moldings, which comprises providing, charging to a mold, and then curing an epoxy resin composition of the invention. In that case, in one preferred embodiment, the epoxy resin composition of the invention is introduced by means of RTM, VARTM or infusion technology into the mold, for curing to form the molding. The mold may include reinforcing materials (e.g. glass fibers or carbon fibers) to produce composites. Preferably, the epoxy resin composition used for such applications is free of solid compounds such as DICY.

The invention particularly provides a process for the production of coatings, which comprises providing, applying to a surface, and then curing an epoxy resin composition of the invention.

It is preferable that the cured epoxy resin is then subjected to thermal post-treatment, for example in the context of the curing process or in the context of optional subsequent heatconditioning.

The invention also provides the cured epoxy resin made of the epoxy resin composition of the invention. In particular, the invention provides cured epoxy resin which is obtainable, or is obtained, via curing of an epoxy resin composition of the invention. The invention in particular provides cured epoxy resin which is obtainable, or is obtained, via the process of the invention for the production of cured epoxy resins.

The epoxy resins cured in the invention have comparatively high glass transition temperature and a comparatively low water up-take.

The epoxy resin compositions of the invention are suitable as coating compositions or impregnating compositions, as adhesive, for the production of moldings in particular of composite moldings using reinforcing fibers (e.g. glass fibers or carbon fibers), or as casting compositions for the embedding, binding, or consolidation of moldings. The epoxy resin composition of the invention are particularly suitable as insulating coatings in electronic applications, e.g. as insulating coating for wires and cable because of the comparatively low content of organic chlorine. The present invention provides the use of N,N′-dialkyl methylcyclohexanediamine as reactive diluent, in particular for epoxy resin compositions with amino hardeners or DICY as curing agent.

The initial viscosity of a curable composition can be determined as mixing viscosity in accordance with the standard DIN ISO 3219 immediately after mixing the components of the curable compositions. The mixing viscosity is determined by the means of a shear stress controlled cone-plate rheometer (e.g. MCR 301, Anton Paar; with a plate and cone diameter of 50 mm, a cone angle of 1° and a gap distance of 0.1 mm). Temperature is a key factor of such measurement because it influences the viscosity and the curing rate of the curable composition. Accordingly, the viscosity needs to be determined at a particular temperature (e.g. 23° C.) in order to allow comparisons.

The glass transition temperature (Tg) can be determined by means of a differential calorimeter (DSC), for example in accordance with the standard ASTM D 3418. A very small amount of specimen (about 10 mg) is heated (e.g. at 20° C./min) in an aluminum crucible, and the heat flux to a reference crucible is measured. This cycle is repeated three times. The glass transition temperature can be determined from the heat-flux curve by way of the inflexion point, or by the half-width method, or by the midpoint temperature method.

The gel time provides, in accordance with DIN 16945 information about the interval between addition of the hardener to the reaction mixture and the conversion of the reactive resin composition from the liquid state to the gel state. The temperature plays an important part here, and the gel time is therefore always determined for a predetermined temperature. By using dynamicmechanical methods, in particular oscillatory rheometry, it is also possible to study small amounts of specimens quasi-isothermally and to record the entire viscosity curve or stiffness curve for these. In accordance with the standard ASTM D4473, the point of intersection of the storage modulus G′ and the loss modulus G″, at which the damping tan δ has the value 1 is the gel point, and the time taken, from addition of the hardener to the reaction mixture, to reach the gel point is the gel time. The gel time thus determined can be considered to be a measure of the hardening rate. The kind of curing and functionality of the epoxy resin and the curing agent play an important part here. E.g. according to the Carothers Equation, the gel point is reached at approximately 75% conversion, when using a di-functional epoxy resin and a tetra-functional amino hardener, or at approximately 83% conversion, when using a di-functional epoxy resin and a tri-functional amino hardener.

The water uptake, in accordance with ISO 62:2008 is a measure for a plastics' tendency to incorporate water. The water uptake is measured as percent of the mass increase after storing the specimen (e.g. the cured epoxy resin) in water for a particular period of time at a particular temperature (e.g. 7 days at 23° C.).

### EXAMPLES

### Example 1: Preparation of N,N′-diisopropyl methylcyclohexanediamine (DIP-MCDA)

730 g (5.7 mol) of a mixture of the isomers 4-methylcyclohexane-1,3-diamine (approx. 80% by weight) and 2-methylcyclohexane-1,3-diamine (approx. 20% by weight) were provided in an autoclave (volume of 3.5 L) with stirring device together with an excess of acetone (1093 g, 18.8 mol). 75 g TiO₂ catalyst and 75 g of a Pd/Ag catalyst supported on Alox have been added within a catalyst cage. The autoclave has been closed and flushed with nitrogen. The reaction mixture was stirred for 4 h at 154° C. Subsequently hydrogen has been added at a pressure of 100 bar and the mixture has been stirred for additional 6 h at 154° C. Reaction water and low boilers has been distilled off in a rotary evaporator at a temperature of 60° C. and a pressure of 30 mbar. The resulting product was a mixture of N,N′-diisopropyl methylcyclohexanediamine (83% by weight) and N-isopropyl methylcyclohexanediamine (17% by weight). To increase the yield of the diisopropyl modified diamine, this reaction product has been mixed again with 1093 g of acetone and the above described procedure has been repeated with fresh catalyst. Now the ratio of N,N′-diisopropyl methylcyclohexanediamine to N-isopropyl methylcyclohexanediamine was >98: 2. DIP-MCDA has been prepared with a selectivity of >95% and a conversion of >99%. The row product has been finally purified by distillation.

### Example 1a: Preparation of N,N′-diisobutyl methylcyclohexanediamine (DIB-MCDA)

DIB-MCDA was prepared is the same way as DIP-MCDA (Example 1), but using an excess of 2-butanone instead of acetone.

### Example 1 b: Viscosity of Mixtures of Epoxy Resin and Modified MCDA

Epoxy resin (bisphenol A diglycidyl ether, BADGE, Epilox A19-03, EEW of 184 g/mol, Leuna Harze) was mixed with DIP-MCDA (Example 1), with DIB-MCDA (Example 1a) and for comparison with N,N′-di(2-ethylhexyl) meta-xylylenediamine (DEH-MXDA; prepared according to US 20150344406, “Amine 1”), each in a weight ratio of 83 parts to 17 parts. Viscosity of the mixtures (mixing viscosity) at a temperature of 23° C. was determined using a conventional shear stress controlled cone-plate rheometer (MCR 301, Anton Paar) with a plate and cone diameter of 50 mm, a cone angle of 1° and a gap distance of 0.1 mm (mixing viscosity). The results are summarized in table 1.

TABLE 1
Viscosity of mixtures of epoxy resin
and modified MCDA and modified MXDA

| Mixture (each with ratio of 83:17 b.w.) | Viscosity (mPas) |
|---|---|
| BADGE + DIP-MCDA | 1769 |
| BADGE + DIB-MCDA | 1753 |
| BADGE + DEH-MXDA | 2456 |

### Example 2: Preparation of Epoxy Resin Compositions

Compositions of epoxy resin (bisphenol A diglycidyl ether, BADGE, Epilox A19-03, EEW of 184 g/mol, Leuna Harze), amine hardener (methylcyclohexyldiamin (MCDA, Baxxodur EC210, BASF)) or diethyltoluenediamine (DETDA, Lonzacure 80, Lonza)) and reactive diluent (N,N′-diisopropyl methylcyclohexanediamine (DIP-MCDA, according to Example 1)), 1,4-butanediol diglycidyl ether (BDGE, Epilox 13-21, EEW of 135 g/mol, Leuna Harze), 1,6-hexanediol diglycidylether (HDGE, Epilox 13-20, EEW of 150 g/mol, Leuna Harze), monoglycidyl ether of C12-C14 aliphatic alcohols (MGE, Epilox 13-18, EEW of 288 g/mol, Leuna Harze), or as a control without reactive diluent)) were prepared using a 1:1 stoichiometric ratio of epoxy groups to NH-functionalities whereas the epoxy groups or the NH-functions of the used reactive diluents were also considered. The epoxy-hardener mixtures were stirred in a propeller mixture at 2000 rpm for 1 min. Detailed amounts of the prepared compositions are summarized in table 2.

TABLE 2
Epoxy resin compositions of the invention (Exp.
1 to 4) and for comparison (Cmp. 1 to 10)

| | BADGE | MCDA | DETDA | DIP-MCDA | BDGE | HDGE | MGE |
|---|---|---|---|---|---|---|---|
| | (g) | (g) | (g) | (g) | (g) | (g) | (g) |
| Exp. 1 | 100 | 14.7 | | 14.7 | | | |
| Exp. 2 | 100 | 18.2 | | 4.5 | | | |
| Exp. 3 | 100 | | 15.6 | 20.5 | | | |
| Exp. 4 | 100 | | 21.8 | 5.9 | | | |
| Cmp. 1 | 100 | 23.7 | | | 14.7 | | |
| Cmp. 2 | 100 | 21.0 | | | 4.5 | | |
| Cmp. 3 | 100 | 21.2 | | | | | 14.7 |
| Cmp. 4 | 100 | 20.7 | | | | | 4.5 |
| Cmp. 5 | 100 | 19.8 | | | | | |
| Cmp. 6 | 100 | | 31.2 | | 20.5 | | |
| Cmp. 7 | 100 | | 25.9 | | 5.9 | | |
| Cmp. 8 | 100 | | 31.2 | | | 20.5 | |
| Cmp. 9 | 100 | | 30.1 | | | | 20.5 |
| Cmp. 10 | 100 | | 24.0 | | | | |

### Example 3: Rheological and Exothermic Profile and Glass Transition Temperature of the Epoxy Resin Compositions

Differential scanning calorimetry (DSC) and rheological experiments were conducted for the epoxy resin compositions of Example 2 immediately following the preparation of the reactive mixtures. DSC was used to determine the reaction and thermal profile (onset temperature (To), peak temperature (Tp), glass transition temperature (Tg)) according to ASTM D 3418 using a heating rate of 20° C./min starting with ambient temperature (23° C.). The results are summarized in table 3.

TABLE 3
Exothermic profile of the epoxy resin compositions

| | | To (° C.) | Tp (° C.) | Tg (° C.) |
|---|---|---|---|---|
| MCDA curing | Exp. 1 | 86 | 119 | 134 |
| | Exp. 2 | 82 | 112 | 152 |
| | Cmp. 1 | 75 | 106 | 144 |
| | Cmp. 2 | 76 | 106 | 158 |
| | Cmp. 3 | 85 | 119 | 135 |
| | Cmp. 4 | 81 | 111 | 148 |
| | Cmp. 5 | 79 | 108 | 169 |
| DETDA curing | Exp. 3 | 121 | 165 | 118 |
| | Exp. 4 | 120 | 159 | 158 |
| | Cmp. 6 | 120 | 158 | 142 |
| | Cmp. 7 | 121 | 158 | 164 |
| | Cmp. 8 | 125 | 164 | 136 |
| | Cmp. 9 | 129 | 166 | 115 |
| | Cmp. 10 | 122 | 158 | 178 |

The initial viscosity (mixing viscosity) at a temperature of 23° C. (for MCDA and DETDA curing) and of 75° C. (for MCDA curing) or 50° C. (for DETDA curing) was determined using a conventional shear stress controlled cone-plate rheometer (MCR 301, Anton Paar) with a plate and cone diameter of 50 mm, a cone angle of 1° and a gap distance of 0.1 mm (mixing viscosity). The rheological profiles (pot life and gel time) at a temperature of 23° C. (for MCDA curing) or 45° C. (for DETDA curing) and of 75° C. (for MCDA and DETDA curing) were determined using a conventional shear stress controlled plate-plate rheometer (MCR 301, Anton Paar) with a plate diameter of 15 mm and a gap distance of 0.25 mm, using rotational mode (pot life) or under oscillatory forces (gel time). The pot life is the time at a given temperature need to attain a viscosity of 6000 mPas. The gel point was defined as the crossover point of the storage and loss moduli and the gel time was defined as the time taken, from addition of the hardener to the reaction mixture, to reach the gel point. The results are summarized in tables 4 and 5.

TABLE 4
Mixing viscosity of the epoxy resin compositions

| | | | at 75° C. (for MCDA |
|---|---|---|---|
| | | | curing) or 50° C. |
| | | at 23° C. | (for DETDA curing) |
| | | mixing | mixing |
| | | viscosity (mPas) | viscosity (mPas) |
| MCDA curing | Exp. 1 | 597 | 67 |
| | Exp. 2 | 895 | 96 |
| | Cmp. 1 | 377 | 62 |
| | Cmp. 2 | 709 | 91 |
| | Cmp. 3 | 305 | 52 |
| | Cmp. 4 | 609 | 83 |
| | Cmp. 5 | 1052 | 111 |
| DETDA curing | Exp. 3 | 1610 | 140 |
| | Exp. 4 | 4350 | 230 |
| | Cmp. 6 | 1280 | 234 |
| | Cmp. 7 | 3720 | 403 |
| | Cmp. 8 | 1200 | 257 |
| | Cmp. 9 | 831 | 176 |
| | Cmp. 10 | 7550 | 803 |

TABLE 5
Rheological profile of the epoxy resin compositions

| | | at 23° C. (for MCDA curing) | | | |
|---|---|---|---|---|---|
| | | or 45° C. (for DETDA curing) | | at 75° C. | |
| | | pot life | gelpoint | pot life | gelpoint |
| | | (min) | (min) | (min) | (min) |
| MCDA | Exp. 1 | 288 | 1738 | 44 | 120 |
| curing | Exp. 2 | 158 | 923 | 24 | 50 |
| | Cmp. 1 | 206 | 830 | 20 | 52 |
| | Cmp. 2 | 140 | 716 | 18 | 43 |
| | Cmp. 3 | 186 | 880 | 28 | 75 |
| | Cmp. 4 | 113 | 568 | 22 | 50 |
| | Cmp. 5 | 160 | 727 | 20 | 41 |
| DETDA | Exp. 3 | 1695 | | 546 | |
| curing | Exp. 4 | 1440 | | 434 | |
| | Cmp. 6 | 1826 | | 457 | |
| | Cmp. 7 | 1550 | | 420 | |
| | Cmp. 8 | 1815 | | 484 | |
| | Cmp. 9 | 2724 | | 672 | |
| | Cmp. 10 | 1370 | | 401 | |

### Example 4: Mechanical Testing of the Cured Epoxy Resin Compositions

Immediately following the preparation of the reactive mixtures, they were degassed at 1 mbar. Subsequently the epoxy resin compositions were cured for 2 h at 80° C. and subsequently 3 h at 125° C. After curing the mechanical tests (tensile modulus (E_t), tensile strength (σ_M), flexural modulus (E_f), flexural strength (σ_fM) were carried out according to ISO 527-2:1993 and ISO 178:2006. The results are summarized in table 6.

TABLE 6
Mechanical properties of the cured epoxy resin compositions

| | | tensile test | | flexural test | |
|---|---|---|---|---|---|
| | | E_t | σ_M | E_f | σ_fM |
| | | (MPa) | (MPa) | (MPa) | (MPa) |
| MCDA | Exp. 1 | 2968 | 82 | 3058 | 123 |
| curing | Exp. 2 | 2883 | 85 | 2955 | 126 |
| | Cmp. 1 | 2833 | 80 | 2934 | 120 |
| | Cmp. 2 | 2835 | 83 | 2927 | 120 |
| | Cmp. 3 | 2921 | 78 | 3026 | 121 |
| | Cmp. 4 | 2848 | 82 | 2940 | 125 |
| | Cmp. 5 | 2894 | 86 | 2889 | 125 |
| DETDA | Exp. 3 | | | 3030 | 119 |
| curing | Exp. 4 | | | 2760 | 110 |
| | Cmp. 6 | | | 2590 | 104 |
| | Cmp. 7 | | | 2470 | 107 |
| | Cmp. 8 | | | 2883 | 116 |
| | Cmp. 9 | | | 2849 | 112 |
| | Cmp. 10 | | | 2760 | 110 |

### Example 4: Determination of the Water Uptake of the Cured Epoxy Resin Compositions

Immediately following the preparation of the reactive mixtures, they were degassed at 1 mbar. Subsequently the epoxy resin compositions were cured for 2 h at 80° C. and subsequently 3 h at 125° C. After curing the water-uptake measurements were carried out according to ISO 62:2008. The water uptake is measured as percent of the mass increase after storing the cured epoxy resins in water for 7 days at 23° C. The results are summarized in table 7.

TABLE 7
Water uptake of the cured epoxy resin compositions

| | |
|---|---|
| MCDA curing | DETDA curing |

| | mass increase (%) | | mass increase (%) |
|---|---|---|---|
| Exp. 1 | 0.31 | Exp. 3 | 0.23 |
| Exp. 2 | 0.33 | Exp. 4 | 0.30 |
| Cmp. 1 | 0.42 | Cmp. 6 | 0.36 |
| Cmp. 2 | 0.39 | Cmp. 7 | 0.33 |
| Cmp. 3 | 0.33 | Cmp. 8 | 0.40 |
| Cmp. 4 | 0.35 | Cmp. 9 | 0.28 |
| Cmp. 5 | 0.30 | Cmp. 10 | 0.33 |

1. A hardener component, comprising:
N,N′-dialkyl methylcyclohexanediamine; and
at least one curing agent,
wherein the at least one curing agents are amino hardeners having at least one primary aliphatic amine group and an NH-functionality of at least 3.

2. The hardener component according to claim 1, wherein a content of the N,N′-dialkyl methylcyclohexanediamine is up to 70% by weight, based on the total amount of N,N′-dialkyl methylcyclohexanediamine and the at least one curing agent of the hardener component.

3. The hardener component according to claim 1, wherein a content of the N,N′-dialkyl methylcyclohexanediamine is from 15 to 60% by weight, based on the total amount of N,N′-dialkyl methylcyclohexanediamine and the at least one curing agent of the hardener component.

4. The hardener component according to claim 1, wherein the at least one curing agents are amino hardeners having two primary aliphatic amino groups.

5. The hardener component according to claim 1, wherein the at least one curing agents are amino hardeners selected from the group of consisting of 2,2-dimethyl-1,3-propanediamine, 1,3-pentanediamine, 1,5-pentanediamine, 1,5-diamino-2-methylpentane, 1,6-hexanediamine, 1,7-heptanediamine, 1,8-octanediamine, 1,9-nonanediamine, 1,10-decanediamine, 1,11-undecanediamine, 1,12-dodecanediamine, 2,5-dimethyl-1,6-hexanediamine, 2,2,4- and 2,4,4-trimethylhexamethylenediamine, dimethyl dicykan, isophoronediamine, diethylenetriamine, triethylenetetramine, aminoethylpiperazine, meta-xylylene diamine, styrene modified meta-xylylene diamine, 1,3-bis(aminomethyl cyclohexane), bis(p-aminocyclohexyl)methane, methylenedianiline, polyetheramines, diaminodiphenylmethane, diaminodiphenylsulfone, 2,4-toluenediamine, 2,6-toluenediamine, methylcyclohexane-1,3-diamine, diethyltoluenediamine, 1,3-diaminobenzene, 1,4-diaminobenzene, diaminocyclohexane, 1,8-menthanediamine, diaminodiphenyl oxide, 3,3′,5,5′-tetramethyl-4,4′-diaminobiphenyl and 3,3′-dimethyl-4,4′-diaminodiphenyl.

6. The hardener component according to claim 1, wherein the N,N′-dialkyl methylcyclohexanediamine is N,N′-dialkyl 4-methylcyclohexane-1,3-diamine of formula I: 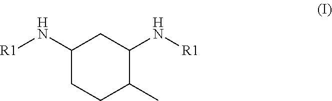
or N,N′-dialkyl 2-methylcyclohexane-1,3-diamine of formula II: 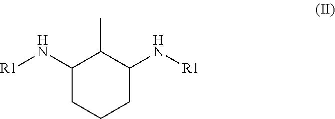
or a mixture thereof,
wherein each R1 is independently of each other an alkyl group having from 1 to 4 carbon atoms.

7. The hardener component according to claim 1, wherein the N,N′-dialkyl methylcyclohexanediamine is N,N′-diisopropyl 4-methylcyclohexane-1,3-diamine, N,N′-diisopropyl 2-methylcyclohexane-1,3-diamine, or a mixture thereof.

8. The hardener component according to claim 1, which further comprises an accelerator for the curing.

9. An epoxy resin composition comprising:
the hardener component according to claim 1; and
a resin component, which comprises at least one epoxy resin.

10. The epoxy resin composition according to claim 9, wherein the at least one epoxy resin is selected from the group consisting of diglycidyl ether of bisphenol A, diglycidyl ether of bisphenol F, diglycidyl ether of hydrogenated bisphenol A, and diglycidyl ether of hydrogenated bisphenol F.

11. The epoxy resin composition according to claim 9, which further comprises additives.

12. A process for the production of cured epoxy resins, the process comprising:
curing an epoxy resin composition according to claim 9.

13. A cured epoxy resin which is obtained by the process according to claim 12.

14. A cured epoxy resin which is obtained by curing of an epoxy resin composition according to claim 9.

15. A process, comprising employing N,N′-dialkyl methylcyclohexanediamine as reactive diluent for epoxy resin compositions.
